(19) United States
(12) Patent Application Publication
(10) Pub. No.: US 2004/0210195 A1
(43) Pub. Date: Oct. 21, 2004

(54) Anti-infection device for endoexo-implants

(76) Inventors: Klaus Affeld, Berlin (DE); Mohammed Bagheri, Berlin (DE)

Correspondence Address: WALKER & JOCKE, L.P.A., 231 SOUTH BROADWAY STREET, MEDINA, OH, 44256, US

(21) Appl. No.: 10/822,214

(22) Filed: Apr. 8, 2004

(30) Foreign Application Priority Data
Apr. 9, 2003 (DE) DE 103 16 726.9

(51) Int. Cl. A61M 5/00

(52) U.S. Cl. 604/171; 604/175

(57) ABSTRACT

An anti-infection device is provided for endoexo implants (1) characterized by a tubular protective membrane (2) which is constructed such that it can be moved out of the sterile region inside the body into the unsterile region outside the body and in this way a wall movement from the inside to the outside, which prevents the penetration of microbes in the opposite direction, can be generated at the point (6) of passage through the skin.

The invention relates to an anti-infection device for endoexo implants.

Endoexo implants are implants which on one side are anchored inside the body and on the other side are outside the body. This means that the endoexo implants penetrate through the skin. Microbes can enter at this point of passage through the skin and move along the surface of the endoexo implant and penetrate inside the body. There is the risk here of infections occurring. This can indeed be controlled within limits by thorough care of the point of penetration through the skin, but endoexo implants must be explanted again and again because of infections.

An example of an endoexo implant is the “fixateur externe” which is employed for stabilizing bone fractures. In this case, a metal screw is screwed into the bone. The other end of the screw, which lies outside the body, is screwed to a bridging rod. Four screws are usually employed per fracture. The bridging rod connects the two ends of the bone separated by the fracture and in this way stabilizes the bone fracture. After the fracture has healed the screws are removed again However, an infection often impedes the healing process. The fact that four screws are employed creases the risk of an infection here.

Another example is an endoexo implant for postoperative care following an amputation. It may be here that the amputation allows only a short stump on to which a conventional arm or leg prosthesis cannot be fixed. The problem can be solved, however, with an endoexo implant. It is anchored with one end in the bone and projects out of the body with the other end. The prosthesis can be fixed to this end. Here also there is the risk of an infection.

A further example is an endoexo implant from the internal medicine sector. Thus, peritoneal dialysis requires a line permanently implanted in the abdominal wall. With one end it lies in the abdominal cavity, and with the other end it projects out of the body. Through this line, the dialysis liquid is passed into the body from the outside and also passed out again. Here also there is the risk of an infection.

A further example is an endoexo implant from the cardiosurgery sector. In most artificial heart assistance systems a supply of pneumatic or electrical energy from the outside is necessary. This is also effected by a line which is connected at one end to the implanted heart assistance system and at its other end to the energy source outside the body. Here also there is the risk of an infection.

In all cases an infection which has penetrated deep into the body requires an operation to remove the endoexo implant.

The infection at an endoexo implant forms first at the boundary at which the three phases of implant, body tissue and germ-containing outside world meet. This is the region where the endoexo implant passes through the skin. The germs can initially be controlled at this point of passage through the skin by frequent cleaning and by external bacteriostatics. However, during a longer indwelling time many germs form a biofilm on the surface of the endoexo implant. This biofilm is a layer of germs which adheres to the channel material and protects itself against the body's defence by a layer of mucus. Since the body cannot penetrate through this biofilm it cannot combat the infection effectively. The biofilm furthermore has the property of extending. It grows in the direction oft he source of nutrients, in this case in the direction of the inside of the body. A pocket thereby forms, which is difficult to clean and which can easily lead to a greater infection

In the laid-open specification DE 37 29 2535 A1, a collar is attached to the endoexo implant in the region of the passage through the skin in order to avoid the risk of infection. This collar is capable of releasing antibiotics and in this way of combating microbes. Experience shows, however, that this device remains effective for only a short time because the microbes develop a resistance.

Devices are furthermore known in which collars have been coated with silver or silver compounds. Here also, however, the development of resistances has been observed.

In the patent specification DE 197 28 489 A1, the catheter surface is coated with an organic fabric of collagen fibres or collagen-polymer fibres. The idea is that the natural collagen has a favourable effect on the growing-in and permanent thriving of the natural body tissue. Experience shows, however, that this measure also cannot prevent infections.

Anti-infection devices are furthermore known in which the action of mechanical forces on the adhering cells is meant to be reduced by a readily movable structure at the point of passage through the skin. This is described in more detail in a work by the Applicant (GroBe-Siestrup, Ch., Affeld, K.: Design criteria for artificial percutaneous devices, Journal of Biomedical Materials Research, vol. 18, 357-382 (1984)). Experience shows, however, that this measure also cannot prevent an infection in the long term.

In the patent specification DE 198 52 848 A1, the catheter surface is surrounded by an anti-infection sheath which is formed in the body and can be pushed out uniformly or in thrusts. However, there is as yet no experience of the effectiveness of this measure. Furthermore, this anti-infection sheath which forms in the body cannot be improved in its biocompatibility using the modem methods of biomaterials technology, such as, for example, plasma treatment. These methods require that the material is brought into a vacuum or exposed to high temperatures.

All these anti-infection devices have the common feature that they cannot prevent an infection and penetration of germs into the body in the long term. They are associated with complications and put the patient at risk.

The invention is based on the object of avoiding the abovementioned disadvantages of the solutions to date and of achieving the object in a technically better manner.

This is achieved in that the endoexo implant 1 has a tubular protective membrane 2 which is fixed on one side 3 hermetically tightly to the endoexo implant 1 and on its other side 4 can be moved from the inside of the body to the outside uniformly or in stages at intervals of time by a device 5. As a result of this, the exogenous material in the form oft he tubular protective membrane 2 moves out of the body at the point 6 of passage through the skin which is at risk of infection A process which takes place naturally in the body, as is the case when non-living natural formations, such as hairs and fingernails, grow out, is thus imitated in a technical manner. The biofilm which forms in the region of the point of passage through the skin is moved out of the body in this way and enters low-nutrient and dry regions, in which it dies. This tubular protective membrane 2 thus surrounds the actual function-bearing element—for example a bone screw—and separates it from the surrounding tissue. The tubular protective membrane 2 is unrolled inside the body like a rolling membrane, or is folded inside the body or can be drawn out from a reservoir inside the body. The length of the foldable protective membrane 2 is in principle limited here, and therefore also the period of time during which it can be drawn out. However, in most uses this is not a disadvantage, because the period of time is in any case limited for other, namely medical, reasons.

Substances which are biologically active and protect the endoexo implant from infections can furthermore be admixed to the material of the tubular protective membrane 2. Thus, antibiotics can be admixed, and then penetrate into the surrounding tissue in the body by diffusion and keep it reliably germ-free. The risk of the abovementioned development of resistance can be counteracted by applying rings of alternating antibiotics. Other substance can promote the adhesion of the cells to the tubular protective membrane. The adhesion of the cells can furthermore be promoted by an increased surface area

The tubular protective membrane 2 which can be moved out of the body steadily or at intervals in the manner described above can be separated of, shortened or also rolled up mechanically—again like the natural formations such as hair and fingernails.

The advantages achieved with the invention are, in particular, that infections can be avoided in the entire region of the endoexo implant 1.

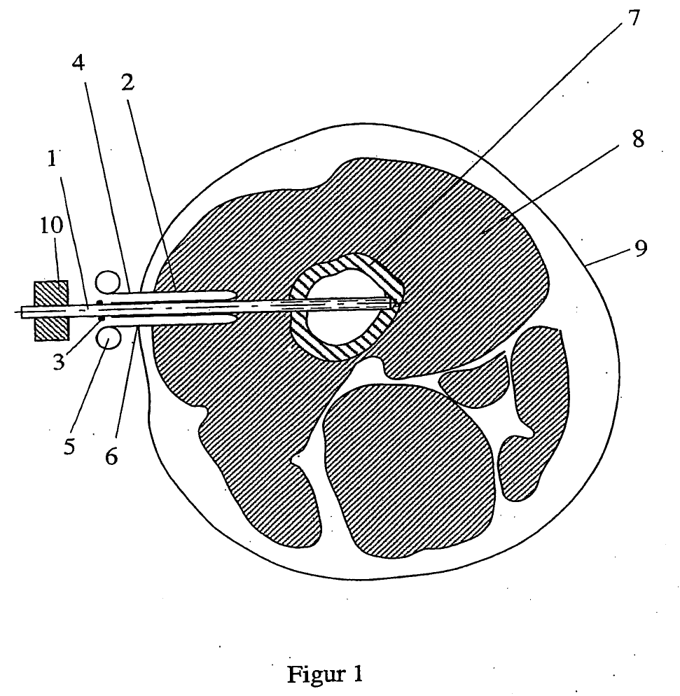

FIG. 1 shows a construction of the anti-infection device for an endoexo implant 1 in a cross-section of a thigh. The endoexo implant 1 here is a bone screw for stabilizing a bone fracture lying outside the plane of the drawing. The screw is screwed into the bone 7 and penetrates through the muscles 8 and furthermore the skin 9 and extends at the point 6 of passage through the skin into the outside region. The entire region inside the body can be considered sterile if the implantation is performed properly. On the other hand, the outside region must be considered unsterile. The tubular protective membrane 2 is fixed hermetically to the screw at the point 3, so that no microbes can penetrate through here. The tubular protective membrane 2 can be moved out of the body by the drawing device 5 and can be unrolled like a rolling membrane. Usually, four bone screws are connected to common bridging rod 10 and stabilize the bone fracture in this manner.

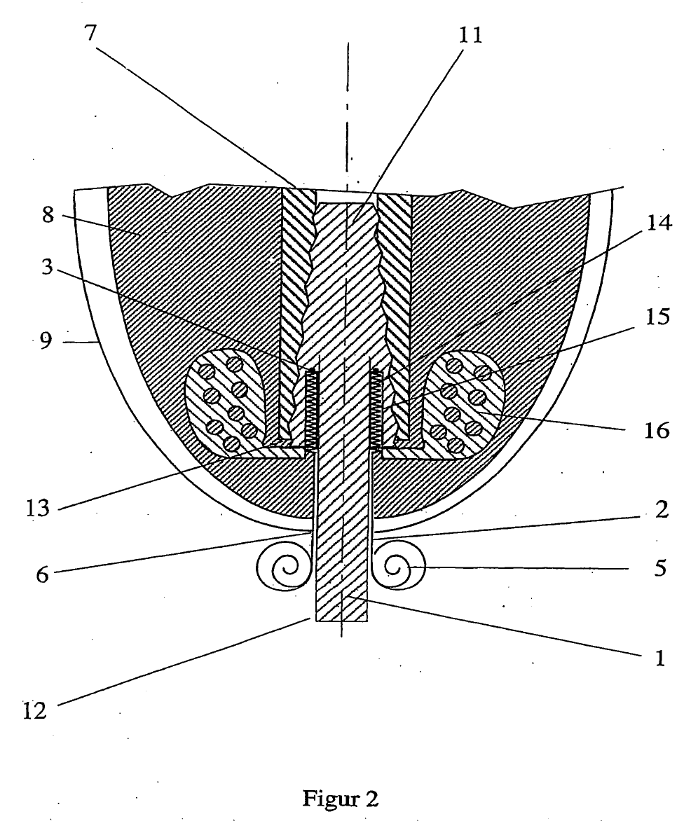

FIG. 2 shows a further endoexo implant in a longitudinal section. This is an endoexo implant for fixing a leg prosthesis to a stump. The endoexo implant 1 here is constructed in several parts in its load-bearing elements. At its proximal end 11 it is firmly connected via a positive lock to the tubular bone 7, and it projects into the outside world with its distal end 12. The tubular bone 7 has been amputated at the point 13. The distal end 2 of the endoexo implant 1 is connected to a prosthesis carrier 11 which penetrates through the skin and is connected to the prosthesis at its end. The endoexo implant 1 is surrounded at its distal end 12 by a tubular protective membrane 2, which is accommodated here in folded form 14 in the recess 15 and therefore extends into the body. The tubular protective membrane 2 is again connected hermetically tightly to the endoexo implant 1 at point 3. The tubular protective membrane 2 is drawn out of the recess 15 at a defined speed by the drawing device 5. At the point 6 of passage through the skin the tubular protective membrane 2 passes through the skin and thus leaves the part of the body. Forces can act at the point 6 of passage through the skin due to the remaining muscles in the stump, and act on the tubular protective membrane 2 which can be grown over by cells. These forces can generate a gap into which microbes can penetrate. To avoid this action of forces, an anchoring 16 is provided. The purpose of this is to take up and lead off into the bone the forces generated by the remaining muscles. The anchoring 16 is inserted through a gap in the tissue during the operation and is then firmly connected mechanically to the endoexo implant 1. Forces which arise are in this way lead off into the bone and cannot act on the point 6 of passage through the skin. For better uptake of the forces, the anchoring 16 is provided with a suitable surface and has breaks through which cells can grow.

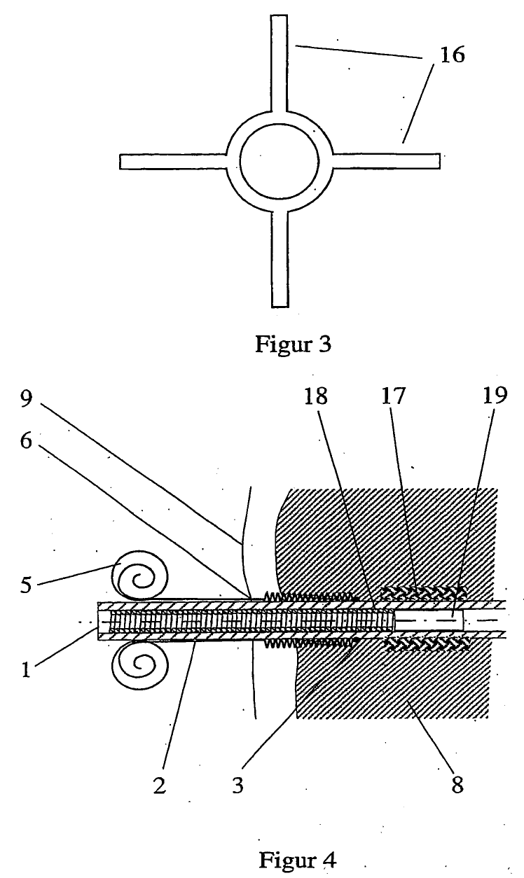

FIG. 3 shows the anchoring 16 in plan view. The anchoring has a plurality of wings on to which tissue can grow. The point 6 of passage through the skin as shown in FIG. 2 is therefore germ-free.

FIG. 4 shows a further endoexo implant 1 in a longitudinal section. This is an endoexo implant from the internal medicine sector. It is a line permanently implanted in the abdominal wall. With one end it lies in the abdominal cavity and with the other end it projects out of the body. Through this line, the dialysis liquid is passed into the body from the outside and also passed out again. The tubular protective membrane 2 is connected hermetically tightly to the line one at point 3 inside the body. The tubular protective membrane 2 surrounds the line 1 in folded form. This has the purpose of keeping the longest possible length of the tubular protective membrane 2 in the sterile region of the body, so that the longest possible functioning time can be achieved. The folding is lifted by the traction of the drawing device 5 and the tubular protective membrane 2 leaves the body at the point 6 of passage through the skin in extended form. The line 1 has a collar 17 through which cells can grow and which fixes it in the tissue or muscle 8. The traction on the tubular protective membrane 2 by the drawing device 5 is transmitted by a flexible but pressure-stiff element 18 and via a rigid tube 19 through the flexible wall of the line 1 to the collar 17 through which cells can grow.

Thus the new anti-infection device and method for endoexo implants achieves one or more of the above stated objectives, eliminates difficulties encountered in the use of prior devices and systems, solves problems and attains the desirable results described herein.

In the foregoing description certain terms have been used for brevity, clarity and understanding, however no unnecessary limitations are to be implied therefrom because such terms are used for descriptive purposes and are intended to be broadly construed. Moreover, the descriptions and illustrations herein are by way of examples and the invention is not limited to the exact details shown and described.

In the following claims any feature described as a means for performing a function shall be construed as encompassing any means known to those skilled in the art to be capable of performing the recited function, and shall not be limited to the features and structures shown herein or mere equivalents thereof. The description of the exemplary embodiment included in the Abstract included herewith shall not be deemed to limit the invention to features described therein.

Having described the features, discoveries and principles of the invention, the manner in which it is constructed and operated, and the advantages and useful results attained; the new and useful structures, devices, elements, arrangements, parts, combinations, systems, equipment, operations, methods and relationships are set forth in the appended claims.

We claim:

1. An anti-infection device comprising a tubular protective membrane in surrounding relation about an endoexo implant, wherein the membrane is constructed such that it can move relative the endoexo implant in an outward direction from a sterile region inside a living body to a unsterile region outside the body, whereby outward movement of a wall of the membrane from the inside region to the outside region is generated at a point of passage of the device through a skin of the body, which movement of the wall prevents penetration of microbes in an opposite inwardly direction.

2. The device according to claim 1, wherein the membrane is connected hermetically tightly to a portion of the endoexo implant, wherein at least one portion of the membrane is compacted, wherein the at least one portion of the membrane is operative to be at least one of unrolled and unfolded.

3. The device according to claim 1, further comprising a drawing device in operative connection with the membrane, wherein the drawing device is operative to draw out the membrane.

4. The device according to claim 3, wherein the drawing device is operative to automatically at least one of continuously and periodically draw out the membrane.

5. The device according to claim 1, wherein the membrane is adapted to facilitate growth thereon of endogenous cells of the body.

6. The device according to claim 1, wherein the membrane includes a biologically active portion.

7. An anti-infection device comprising:
an endoexo implant adapted for mounting to an interior portion of a living body; and
a flexible tubular protective membrane in surrounding relation about the endoexo implant, wherein the membrane includes a first end and a second end, wherein the first end of the membrane is connected hermetically tightly to a portion of the endoexo implant, wherein at least one portion of the membrane is compacted between the first end and the second end, wherein when the device is mounted to an interior portion of a living body with the at lease one portion of the membrane positioned within the body and the second end of the membrane positioned outside the body, the at least one portion of the membrane is operative to elongate and move with respect to the endoexo implant in a direction that extends outwardly of the body.

8. The device according to claim 7, further comprising a drawing device in operative connection with the second end of the membrane, wherein the drawing device is operative to urge the at least one portion of the membrane to elongate and move with respect to the endoexo implant in the outwardly direction.

9. The device according to claim 8, wherein the drawing device is operative to periodically urge the at least one portion of the membrane to move with respect to the endoexo implant.

10. The device according to claim 7, wherein the at least one portion of the membrane is at least one of folded or rolled.

11. The device according to claim 7, wherein the membrane includes a surface with an antibiotic thereon.

12. The device according to claim 7, wherein the endoexo implant includes a bone screw.

13. The device according to claim 7, wherein the endoexo implant includes a hollow line which is operative to permit fluids to pass therethrough.

14. The device according to claim 7, further comprising an anchor in operative connection with the endoexo implant, wherein the anchor includes a plurality of wings which extend radially outwardly from the endoexo implant.

15. A method comprising:
a) mounting an endoexo implant to an interior portion of a living body, wherein a tubular protective membrane is in operative surrounding connection about a portion of the endoexo implant, wherein at least one portion of the membrane positioned within the interior of the body is in a compacted state; and
b) urging the least one portion of the membrane to move with respect to the endoexo implant in a direction outwardly of the body, wherein the at least one portion of the membrane elongates.

16. The method according to claim 15, wherein in (a) the endoexo implant is mounted to a bone of the body.

17. The method according to claim 16, wherein in (a) the endoexo implant includes a bone screw.

18. The method according to claim 15, wherein (b) includes moving the second end of the membrane with a drawing device positioned outside the body.

19. The method according to claim 18, wherein in (b) the drawing device periodically urges the at least one portion of the membrane to move in the outwardly direction.

20. The method according to claim 15, wherein in (a) the compacted state includes the at least one portion of the membrane being at least one of folded and rolled up, wherein (b) includes at least one of unfolding and unrolling the at least one portion of the membrane.

21. The method according to claim 15, wherein (b) includes moving the at least one portion of the membrane from inside the body to outside the body.

22. The method according to claim 15 wherein in (a) the membrane includes a surface with an antibiotic thereon.

23. The method according to claim 15, wherein in (a) the endoexo implant includes a hollow line; and further comprising:
(c) moving a dialysis liquid through the line.

24. The method according to claim 15, wherein in (a) an anchor is in operative connection with the endoexo implant, wherein the anchor includes a plurality of wings which extend radially outwardly from the endoexo implant.

25. The method according to claim 15, wherein in (a) the membrane is connected hermetically tightly to a portion of the endoexo implant located inside the body.
